(19) United States
(12) Patent Application Publication
(10) Pub. No.: US 2026/0328743 A1
(43) Pub. Date: Oct. 22, 2026

(54) METHOD OF QUANTIFYING AND ANALYZING FLUORINE FROM SEMICONDUCTOR PROCESS EMISSIONS

(71) Applicant: Applied Materials, Inc., Santa Clara, CA (US)

(72) Inventors: Joseph VAN GOMPEL, Santa Clara, CA (US); Benjamin GROSS, Santa Clara, CA (US); Andy NGUYEN, Santa Clara, CA (US)

(21) Appl. No.: 19/646,331

(22) Filed: Apr. 13, 2026

Related U.S. Application Data
(60) Provisional application No. 63/790,652, filed on Apr. 17, 2025.

(51) Int. Cl. G01N 33/00; H01J 49/00; H01J 49/42

(52) U.S. Cl. CPC G01N 33/0008; G01N 33/0036; H01J 49/0009; H01J 49/4215

(57) ABSTRACT

The present disclosure generally relates to a method for calibrating a sensor for detecting fluorine gas in an exhaust of the plasma processing system. The method begins by introducing a quantity of fluorine containing gas from a source into a processing chamber exhaust system. The fluorine containing gas is dissociated. The level of fluorine (F₂) between a pump and a scrubber is measured with a sensor. A calibration curve is calculated for the sensor to determine quantities of F₂ after the pump based on the amount of introduced fluorine containing gas. The sensor is calibrated to sense an amount of F₂ in the exhaust system based on the calibration curve.

### CROSS-REFERENCE TO RELATED APPLICATION

This application claims priority to the U.S. Provisional Patent Application Ser. No. 63/790,652 filed Apr. 17, 2025 of which is incorporated herein by reference in its entirety.

### BACKGROUND

### Field

Embodiments of the present disclosure generally relate to semiconductor processing equipment. More particularly, embodiments of the present disclosure relate to the quantifying the amount of fluorine gas in the exhaust of the semiconductor processing equipment.

### Description of the Related Art

The process gases used by semiconductor processing facilities include many compounds, such as fluorine and chlorine gases. Typically, fluorine and chlorine may be used in semiconductor processing for chamber cleaning to prevent contamination built up in the processing chamber from affecting the quality of the devices built on a wafer. A remote plasma source may be coupled to the processing chamber to provide a plasma with the fluorine and chlorine for chamber cleaning. The fluorine and chlorine compounds then exit the processing chamber.

However, the fluorine and chlorine compounds must be abated or treated before disposal, due to regulatory requirements and environmental and safety concerns. Fluorine gas (F₂) is very corrosive and toxic, and can contribute to noxious odors outside the fab. F₂ is not visible by Fourier transform infrared spectroscopy (FTIR), the conventional analysis technique of choice, and results in F₂ being overlooked in most process emissions analyses. Current state-of-the-art for F₂ analysis involves a fluorine electrochemical sensor which must be calibrated, typically using tanks of F₂. However, it is difficult to obtain safety approval to bring F₂ tanks into the fab. In addition, the concentration of F₂ levels for some processes (e.g. CVD) may be impossible to obtain when relying on these techniques because F₂ in tanks is usually diluted to 10-20% in nitrogen.

Accordingly, what is needed in the art is an improved technique for detecting F₂ in processing chamber exhaust systems for abating the compounds produced in semiconductor processes.

### SUMMARY

The present disclosure generally relates to a method for calibrating a sensor for detecting fluorine gas in an exhaust of the plasma processing system. The method begins by introducing a known quantity of fluorine containing gas from a source into a processing chamber exhaust system. The fluorine containing gas is dissociated in the processing chamber exhaust system. The level of fluorine (F₂) between a pump and a scrubber processing chamber exhaust system is measured with a sensor. A calibration curve is calculated for the sensor to determine quantities of F₂ after the pump based on the amount of introduced fluorine containing gas. The sensor is calibrated to determine an amount of F₂ in the exhaust system based on the calibration curve and the flow of fluorine containing gas into the processing chamber exhaust system.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a front schematic view of a plasma processing system having a gas delivery assembly.

FIG. 2 illustrates a process flow for reducing fluorine gas an exhaust of the plasma processing system.

FIG. 3 illustrates a process flow for calibrating a sensor to detect fluorine gas in the exhaust of the plasma processing system.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments disclosed herein include a remote plasma source coupled to a processing system and/or abatement system and a method for determining fluorine concentration levels in the exhaust system. The remote plasma source is coupled to a gas delivery assembly and may be deployed in semiconductor processing systems such as etch chambers, plasma treatment chambers, physical vapor deposition chambers, chemical vapor deposition chambers, high density plasma chemical vapor deposition (HDPCVD) chambers, low-pressure chemical vapor deposition (LPCVD) chambers, among others. The gas delivery system is suitable for providing gases including fluorine or fluorine containing gas, as well as other processing, cleaning or abatement gases. The process precursor gases and/or foreline plasma abatement system may be used to generate F₂ calibrations to quantify F₂ in the emissions for semiconductor process such as etch or CVD. The RPS may be used for F₂ generation in situ for calibrating fluorine electrochemical sensors.

F₂ can be generated in situ without the need of bringing in tanks of F₂ into the fab. In one example, fluorine containing gas, such as NF₃ among others, is flowed from a processing chamber, and/or a remote plasma source coupled to the processing chamber, and/or a fluorine containing gas source coupled to the processing chamber exhaust system, is used to generate F₂. In some examples, a foreline plasma abatement system is used to convert fluorine containing gas exiting the processing chamber or injected into the processing chamber exhaust system to fluorine radicals, which the recombine in the exhaust system to F₂. Use of the foreline plasma abatement system minimizes/eliminates any damage to the processing chamber from direct exposure to F₂. Tests have indicated that, in addition to NF₃ gas, CF₄+O₂, SF₆+O₂, and C₄F₈+O₂ gas all can be used to generate F₂. Other fluorine containing gases may also be used to generate F₂.

The hardware for generating the F₂ may be a processing chamber with an abatement system installed in a foreline of a chamber exhaust system for fluorine greenhouse gas (F-GHG) abatement. The appropriate fluorine containing gas is flowed from the processing chamber and is dissociated using the abatement system in the foreline to optimize F₂ generation. Thus, no gas bottles containing F₂ are needed. A sensor can monitor known quantities of F₂ at different locations in the abatement of the effluent to develop a calibration model, such as a curve, for the sensor from which the amount of fluorine in the exhaust system can be derived from the measured fluorine detected by the sensor, the amount of fluorine containing gas entering the foreline, and the calibration model.

Changing the total flow of the fluorine containing gases allows for rapid concentration change. For example:

2NF₃-->3F₂+N₂

CF₄+O₂-->F₂+COF₂.

In each of the above equations, F₂ is formed in real time. In addition to avoiding the paperwork of getting an F₂ tank into the fab, the F₂ formed in this process already has an existing abatement mechanism so the F₂ formed is readily and safely treated in real time. This enables the use of process precursor gases and foreline plasma abatement to generate F₂ calibrations and quantify F₂ in the emissions of semiconductor process such as etch and CVD. By knowing the quantity of F₂ in the semiconductor process emission, proper abatement can be put into place for reducing F₂ emissions, and/or for environmental reporting purposes.

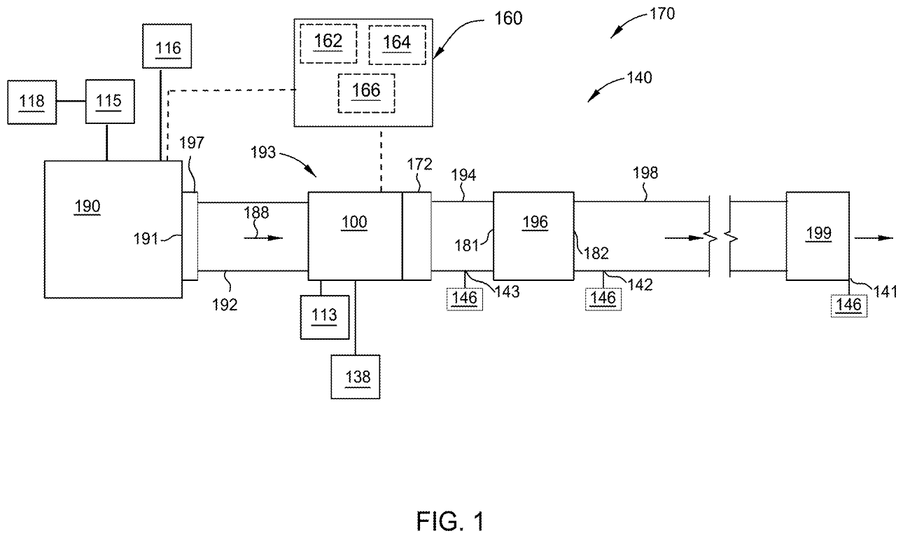

FIG. 1 is a schematic side view of a vacuum processing system 170 having vacuum processing chamber 190 fluidly coupled to an exhaust system 140. The vacuum processing system 170 includes at least one vacuum processing chamber 190.

The vacuum processing chamber 190 is generally configured to perform at least one integrated circuit manufacturing process, such as a deposition process, an etch process, a plasma treatment process, a pre-clean process, an ion implant process, or other integrated circuit manufacturing process. The process performed in the vacuum processing chamber 190 may be plasma assisted. For example, the process performed in the vacuum processing chamber 190 may be a plasma deposition process for depositing a material on a substrate or a plasma etch process for removing a material from a substrate. During processing and cleaning, gases are introduced into the vacuum processing chamber. Certain operations, such as deposition or etch, have gases ionized to form a plasma for processing a substrate in a vacuum environment. For example, the vacuum processing chamber 190 may maintain a pressure of less than about 0.5 Torr to about 500 Torr, such as about 3 Torr to about 80 Torr, such as about 3 Torr to about 60 Torr, such as about 3 Torr to about 50 Torr, such as about 3 Torr to about 40 Torr, or such as about 3 Torr to about 5 Torr during processing.

The vacuum processing chamber 190 is coupled to a gas source 116. The gas source 116 may provide process or precursor gases into the vacuum processing chamber 190. The gas source 116 may provide a cleaning gas to the vacuum processing chamber 190. For example, the gas source 116 may provide a fluorine based gas to the vacuum processing chamber 190. In one example, the gas source 116 provides NF_{3,} CF₄+O₂, SF₆+O₂, and/or C₄F₈+O₂, among other gases into the vacuum processing chamber 190.

The vacuum processing chamber 190 may optionally be equipped with a remote plasma source (RPS) 115 coupled to a gas source 118. The gas source 118 may provide a cleaning gas to the RPS 115. For example, the gas source 118 may provide a fluorine or chlorine based gas to the RPS 115. The RPS 115 forms a plasma from the cleaning gas to clean the vacuum processing chamber 190. In one example, the gas source 118 provides F₂ to the RPS 115, which generates fluorine radicals that flow into the vacuum processing chamber 190 for cleaning residues and contamination from surfaces inside the vacuum processing chamber 190.

The gases used in the cleaning of the vacuum processing chamber 190 and their byproducts, create potent and long lasting effluent which may be dangerous to humans and/or the environment. For example, one or more process gases may contain fluorine or chlorine which combine with other gases or byproducts to form a fluorinated gas or chlorinated gas which is evacuated from the vacuum processing chamber 190 as effluent to stop processing and reduce chamber contamination and/or substrate defects.

The vacuum processing chamber 190 has a chamber exhaust port 191. The vacuum processing chamber 190 is coupled through the chamber exhaust port 191 to the exhaust system 140. The exhaust system 140 includes a processing chamber exhaust foreline 192 and may have a turbo pump 197. The chamber exhaust port 191 is coupled to the processing chamber exhaust foreline 192. The chamber exhaust port 191 may optionally be coupled directly to the turbo pump 197. The effluent is moved from the vacuum processing chamber 190 into the processing chamber exhaust foreline 192. The effluent in the processing chamber exhaust foreline 192 remains at a low vacuum pressure such as below 500 Torr.

The exhaust system 140 may include a gas source 138. The gas source 138 may be suitable for providing gases for abating effluent in the exhaust system 140. In one example, the gas source 138 provides oxygen, nitrogen, H₂O or other gas to the exhaust system 140. The gas source 138 may be fluidly coupled to the processing chamber exhaust foreline 192 or other suitable location in the exhaust system 140.

The exhaust system 140 may optionally include an abatement system 193 having an abatement reactor 100. The abatement system 193 may optionally have a particle collection trap 172. The abatement reactor 100 of the abatement system 193 may optionally be fluidly coupled to the vacuum processing chamber 190 by the processing chamber exhaust foreline 192. The gas source 138 may be fluidly coupled to the abatement reactor 100 to provide a gas used abating the effluent in the plasma in the abatement reactor 100.

In one example, the abatement reactor 100 may be a plasma abatement reactor. In another example, the abatement reactor 100 may be a thermal abatement reactor. The abatement reactor 100 breaks down the effluent into less dangerous or more easily disposed of chemicals. Hydrogen-based reagents may be added to the processing chamber exhaust foreline 192 or the abatement reactor 100 to aid in breaking down the effluent into chemicals which may be further processed or disposed. The reagents may include hydrogen (H₂), ammonia (NH₃), methane (CH₄) water (H₂O) or other suitable hydrogen containing gas. In one example, the hydrogen based reagent is water, H₂O. After abatement by the abatement reactor 100, the effluent may contain hydrogen fluoride (HF) and/or hydrogen chloride (HCl). In one example, no effluent is provided to the abatement reactor, while a fluorine containing gas, such as F₂, to the abatement reactor 100.

The optional particle collection trap 172 may be located downstream of the abatement reactor 100 in order to collect particles formed in the abatement reactor 100 and to cool the exhaust exiting the abatement reactor 100. The particle collection trap 172 may be directly coupled to the abatement reactor 100. Alternately, a conduit may couple the particle collection trap 172 to the abatement reactor 100.

An abatement exhaust foreline 194 is fluidly coupled to a downstream side of the abatement reactor 100. Alternately, the abatement exhaust foreline 194 is fluidly coupled to a downstream side of the particle collection trap 172. The first fluid conduit is fluidly coupled to a pump 196.

The pump 196 has an inlet 181 and an outlet 182. The pump 196 pulls the effluent from the vacuum processing chamber 190 through the abatement system 193 into the inlet 181 of the pump 196. The pump 196 may be a vacuum pump or rough pump. The pump 196 operates in a vacuum range having a pressure between about 1×10⁻³ Torr to approximately 1×10⁻² Torr. The pump 196 may routinely operate in this range when no gases are flowing into chamber 190. The pressure will increase when chamber 190 and reactor 100 is in operation, typically the pressure will be no higher than about 1.0 Torr. In one example, pump 196 is a rough pump suitable for working in the vacuum range. As such, the pump 196 pulls a vacuum from the vacuum processing chamber 190, through the abatement system 193, to the inlet 181 in the pump 196. For example, the pump 196 may maintain a vacuum on the abatement system 193 of around 10⁻³ Torr upstream of the pump 196, i.e., between the abatement reactor 100 and the inlet 181 of the pump 196.

The outlet 182 of the pump 196 is fluidly coupled to a pump exhaust foreline 198. The pump exhaust foreline 198 receives the treated effluent from the abatement system 193 via the pump 196. The treated effluent in the pump exhaust foreline 198 is at a pressure of about 1 (atm) atmospheric pressure, i.e., 760 Torr. Thus, the treated effluent may go from a vacuum pressure of about 10⁻³ Torr in the abatement exhaust foreline 194 to atmospheric pressure of about 760 Torr in the pump exhaust foreline 198.

The fluorinated gas and/or chlorinated gas may be treated by a scrubber 199 coupled to the pump exhaust foreline 198. To properly treat the fluorinated gas and/or chlorinated gas at the scrubber 199, the amount of fluorine and/or chlorine containing gas must be quantified. In some examples, where the effluent contains a fluorinated gas, the hydrogen based reagents create gaseous hydrogen fluoride (HF) which dissolves in water to yield an aqueous hydrofluoric acid. In other examples, where the effluent contains a chlorinated gas, the chlorine based reagents create gaseous hydrogen chloride (HCl) which dissolves in water to yield an aqueous hydrochloric acid. It has been found that monitoring the F₂ gas after the pump 196, or in the pump exhaust foreline 198, and/or the scrubber 199, may be suitable to determine the amount of fluorine or chlorine containing gas reaching the scrubber 199. In one example, gas sensor 146 may be fluidly coupled to the pump exhaust foreline 198 downstream of the outlet 182 of the pump 196. The process emission can be quantified through the use of the gas sensor 146. An emissions calculation for fluorine or chlorine containing effluent can be tabulated to provide suitable abatement, effluent monitoring, and regulatory reporting purposes. The sensor 146 is suitable for mass spectrometry. For example, the sensor 146 may be a Faraday cup, ion-to-photon detectors, tandem mass spectrometer a quadrupole mass analyzer or other suitable sensor. QMS (quadrupole mass spectrometry) is well-suited to F₂ analysis. Another device that may be used as the sensor 146 is a Residual Gas Analyzer, or RGA. In most applications, an RGA would be located in the foreline 194 for example at location 143. The technology used in an RGA is similar to that in a QMS, with the only significant distinction being that RGAs are typically used to sample at vacuum pressure whereas QMSs are typically used to sample at atmospheric pressure. The sensor 146 may be a QMS sensor consisting of four cylindrical rods creating an oscillating electric field that allows ions with specific mass to charge (m/z) values to pass while filtering out other ions. For example, the QMS sensor 146 may be configured to select an ion with a particular m/z or allows the operator to scan for a range of m/z-values by continuously varying the applied voltage

The sensor 146 may be placed in several locations for detecting F₂. For example, the sensor 146 may additionally be located prior to each/any point of use abatement unit (location 143), pre-scrubber or just before the scrubber 199 (location 142), and post-scrubber just after the scrubber 199 (location 141). F₂ is difficult to remove in rooftop scrubber with emissions resulting in violations and bad smells outside and around the fab. The QMS sensor 146 allows for the detection of the F₂ but requires calibration. Thus, prior to operating the QMS sensor 146 in a production capacity, the sensor 146 can be calibrated using the known quantity of fluorine containing gas used to generate the F₂ to generate a calibration model that includes the sensed fluorine and quantity of fluorine containing gas as inputs to yield the amount of fluorine present in the exhaust system and ultimately reaching the scrubber.

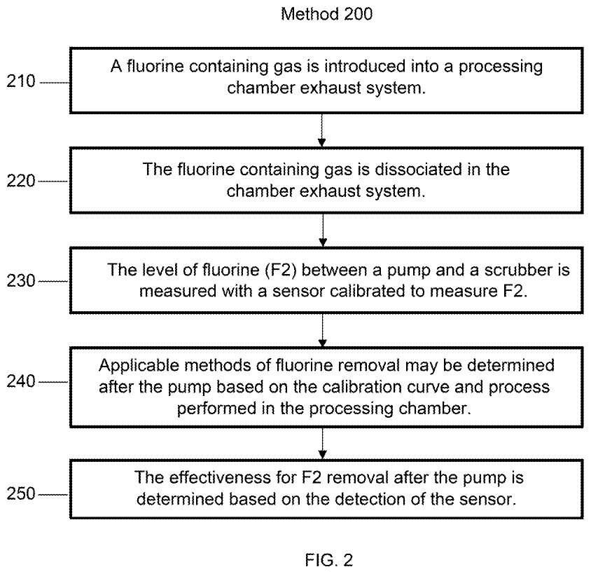

A system controller 160 is coupled to the vacuum processing system 170 for controlling the vacuum processing chamber 190, the abatement system 193, pump 196 and/or gas sensor 146. For example, the system controller 160 may be in communication with the gas sensor 146 and control the operations of the RPS 115, gas source 116 and/or abatement reactor 100 to calculate a schedule for abating certain gases, such as fluorine, chlorine or halogen bromine Br2. Whereas fluorine and chlorine may be a process or cleaning gas used in the processing chamber 190, halogen bromine Br2 may be formed as a byproduct for some processes that use hydrogen bromide as one of the process gases. In operation, the system controller 160 enables a process flow 200 illustrated in FIG. 2 for quantify the F₂ in process emissions for treatment.

The system controller 160 generally includes a central processing unit (CPU) 162, memory 164, and support circuits 166. The CPU 162 may be one of any form of a general purpose processor that can be used in an industrial setting. The memory 164, non-transitory computer-readable medium, or machine-readable storage device, is accessible by the CPU 162 and may be one or more of memory such as random access memory (RAM), read only memory (ROM), floppy disk, hard disk, or any other form of digital storage, local or remote. The support circuits 166 are coupled to the CPU 162 and may comprise cache, clock circuits, input/output subsystems, power supplies, and the like. The system controller 160 is configured to perform the process flow 200 stored in the memory 164. The various implementations disclosed in this disclosure may generally be implemented under the control of the CPU 162 by executing computer instruction code stored in the memory 164 (or in memory of a particular processing chamber) as, e.g., a computer program product or software routine. That is, the computer program product is tangibly embodied on the memory 164 (or non-transitory computer-readable medium or machine-readable storage device). When the computer instruction code is executed by the CPU 162, the CPU 162 controls the instructions to the gas sources and gas sensors for controlling the abatement of effluent downstream of the pump exhaust foreline 198 as described below in accordance with the various implementations.

Fluorine gas is very corrosive and toxic, and can contribute to noxious odors outside the fab. There are other ways of monitoring F₂, but these require tanks of fluorine gas to be brought into the fab. It is difficult to obtain safety approval to bring F₂ tanks into the fabrication facility. In addition, the concentration required for F₂ levels on some processes (e.g. CVD) may be impossible to obtain because F₂ in tanks is always diluted to 10-20% in nitrogen. Foreline plasma from NF₃ enables full N₂ and F₂ concentration. QMS sensors are provided for monitoring the F₂ during processing. However, QMS sensors need to be calibrated to understand the amount of the material detected. In this case, the QMS sensor is calibrated to detect F₂. Alternatively, calibration may be done off-site immediately before or after testing in the fab, though this is not optimal as QMS sensors, or RGA sensors may drift when turned off and on during the period between sampling and calibration.

FIG. 2 illustrates a method 200 for detecting fluorine gas (F₂) in an exhaust system of a processing chamber. In a first operation 210, a fluorine containing gas is introduced into a processing chamber exhaust system. The fluorine containing gas may be a process gas or a cleaning gas evacuated from the processing chamber. For example, the fluorine containing gas may be NF₃ used in the cleaning of the processing chamber. The fluorine containing gas is provided in a known quantity based on process parameters set recipes for processing and/or cleaning. The fluorine is harmful to human health and the environment. Therefore, the fluorine is abated prior to being released into the environment.

In a second operation 220, the fluorine containing gas is dissociated in the chamber exhaust system. The chamber exhaust system may have a foreline abatement system. The foreline abatement system may use plasma, heat or other treatment process for breaking the fluorine containing gas apart, or disassociating the gas, for treatment. For example, a plasma reactor may introduce H₂O for forming HF which can be removed by a wet scrubber. However, F₂ is not easily removed by a scrubber and may pass through.

In a third operation 230, the level of fluorine between pump and scrubber is measured. The QMS (quadrupole mass spectrometry) sensor is well-suited to F₂ analysis but requires calibration.

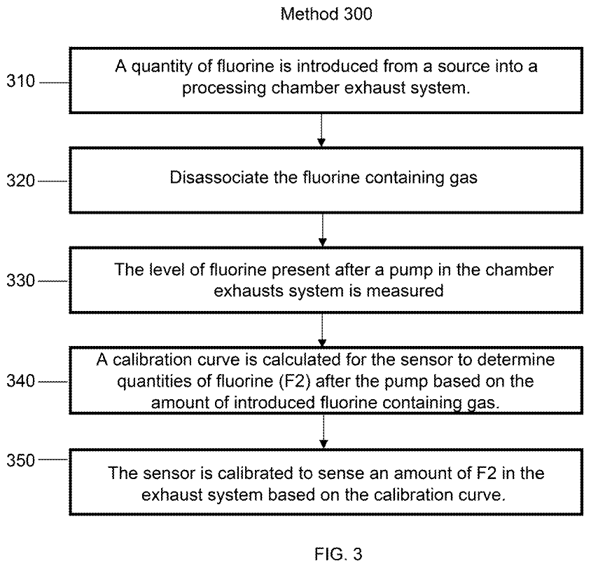

Turning briefly to FIG. 3, a method for calibrating a sensor for detecting fluorine gas in an exhaust of a plasma processing system is illustrated. To calibrate the sensor, fluorine is provided to the sensor for data collection and developing a calibration curve/model.

In a first operation 310, a known quantity of fluorine containing gas is introduced from a fluorine containing gas source into a processing chamber exhaust system. F₂ can be provided to the processing chamber exhaust system in several different ways. In one example, fluorine containing gas, such as from a cleaning gas and/or processing gas used in the processing chamber is flowed from the processing chamber into the chamber exhaust system. Alternatively, a fluorine containing gas may be injected into the chamber exhaust system downstream of the processing chamber. A remote plasma source for cleaning coupled to the chamber may be used to generate the F₂ by disassociating a cleaning gas such as NF₃ inside the chamber prior to the F₂ entering chamber exhaust system.

In a second operation 320, the fluorine containing gas is dissociated to ultimately form F₂. In another example, a foreline plasma abatement system may be used to convert the fluorine containing gas source present in the processing chamber exhaust system to F₂. For example, NF₃ from the processing chamber may be converted to F₂ +N₂. The foreline plasma abatement system generally dissociates the fluorine containing gas to include fluorine radials, such recombine in the exhaust system as F₂. Tests have indicated that, in addition to NF₃, CF₄+O₂, SF₆+O₂, and C₄F₈+O₂ all be used can generate F₂ using the foreline abatement system. Once the fluorine containing gas is dissociated, care is taken to ensure the formation of F₂ is preferentially formed over other fluorinated compounds. For example, other gases such as hydrogen may be turned off so that the amount of hydrogen is reduced or eliminated from the chamber exhaust while F₂ is formed.

In a third operation 330, the level of fluorine between a pump and a scrubber is measured with a sensor. Performance of the sensor, QMS detector and sampling lines, is aided by passivation to obtain an accurate F₂ signal. The passivation ensures F₂ ions have a specific mass-to-charge ratio (m/z) values for detection by the sensor. This pre-test step also enables a good calibration to quantify F₂.

Changing the total flow of the fluorine gas allows for rapid concentration change. In one example, ammonia or tetrafluoromethane may be used to generate F₂.

2 NF₃-->3F₂+N₂.

CF₄+O₂-->F₂+COF₂

In each of the above equations, F₂ is formed in real time with the amount detected by the sensor.

In a fourth operation 340, a calibration curve is calculated for the sensor to determine quantities of fluorine after the pump based on the amount of introduced fluorine containing gas. The known amount of F₂ from the previous operation is plotted against the instrumentally detected mass scale. The calibration curve depicts a relationship between the instrumental mass scale and the known m/z values of the F₂. This calibration process enables both identifying the F₂ from other analytes, and for measuring the abundance of F₂.

In a fifth operation 350, the sensor is calibrated to sense an amount of F₂ in the exhaust system based on the calibration curve and the amount of fluorine containing gas flowing in the exhaust system. In this manner, emissions can be calculated by weight/wafer for all greenhouse gases (GHG), including byproducts, such as F_{2,} and integrate to ensure accurate total global warming potential (GWP) per wafer.

Turning back to FIG. 2, in a fourth operation 240, the level of fluorine (F₂) after the pump is measured with the sensor, the sensor calibrated using a calibration model generated using F₂ formed in situ in the chamber exhaust system. The sensor may additionally or alternately be disposed prior to or just after the scrubber. F₂ is not directly water soluble, but slowly converts to HF in the presence of moisture. So as F₂ gas moves down the exhaust ducts, some portion of it will convert to HF. When F₂ reaches the scrubber, such as a wet scrubber at the end of the chamber exhaust system, even more will convert to HF, but some portion will pass through as F₂ and be emitted from the fab. The sensor can detect the F₂ of concern passing through the scrubber.

In a fifth operation 250, the effectiveness for F₂ removal after the pump is determined based on the detection of the sensor. The calibration curve and process performed in the processing chamber enable the calibrated sensor to provide real-time information for the amount of F₂. Applicable methods of fluorine removal may be determined after the pump based on the detected F₂. With the QMS sensor able to detect known quantiles of F₂ during different stages of the exhaust system, changes can be made to ensure adequate F₂ is abated prior to the scrubber. Abatement processes or scrubber capacity may be modified to adequately remove all F₂, regardless of effluent composition. For example, a point of use scrubber may be added to the exhaust system for abating F₂. Furthermore, the post scrubber sensor can be used to provide required environmental reporting information.

If all of the F₂ converts to HF before or at the house scrubber, then the acid load at the house scrubber is exactly the same as if the abatement system is used and HF is immediately produced. The fluorine atoms come out in the house scrubber and are emitted. Therefore, if the house scrubber does not have capacity to remove all F₂, then effectiveness information may be used to determine when to add Point-of-Use Abatement (POU) and/or area scrubber along with a plasma abatement system in the chamber exhaust system.

Advantageously, a sensor can be calibrated and used for real-time analysis of F₂ gas in processing effluent. The method disclosed allows for the avoidance of getting F₂ gas tanks into the fab when calibrating the F₂ sensor to effect the substantial removal of F₂ outside emissions. In addition to avoiding the problems of getting an F₂ tank into the fabrication facility, the F₂ formed in this process already has an existing abatement mechanism so the F2 formed is readily and safely treated in real time.

While the foregoing is directed to the preferred embodiment of the present invention, other and further embodiments of the invention may be devised without departing from the basic scope thereof, and the scope thereof is determined by the claims that follow.

What is claimed is:

1. A method for calibrating a sensor for detecting fluorine gas in an exhaust of a plasma processing system, the method comprising:
introducing a quantity of fluorine containing gas from a source into a processing chamber exhaust system;
disassociating the fluorine containing gas to form fluorine (F₂);
measuring a level of fluorine (F₂) between a pump and a scrubber with a sensor;
generating a calibration curve for the sensor indicative of a quantity of fluorine (F₂) present between the pump and the scrubber based on the quantity of fluorine containing gas introduced to the processing chamber exhaust system; and
calibrating the sensor to relate the sensed amount of fluorine (F₂) in the exhaust system to a concentration of fluorine containing gas based on the calibration curve.

2. The method of claim 1, wherein the sensor is a quadrupole mass spectrometry sensor.

3. The method of claim 1, wherein fluorine (F₂) is generated in the processing chamber exhaust system from the fluorine containing gas.

4. The method of claim 3, wherein the source is a remote plasma source (RPS) for cleaning coupled to the plasma processing system and the RPS is configured to supply the fluorine containing gas.

5. The method of claim 1, wherein a plasma abatement system is disposed in the chamber exhaust system and converts NF₃, the fluorine containing gas, to F₂+N₂.

6. The method of claim 1 further comprising:
passivating fluorine (F₂) ions to have a specific mass-to-charge ratio (m/z) values for detection by the sensor.

7. The method of claim 1, wherein the calibration curve comprises:
plotting a known amount of fluorine (F₂) against a mass scale detected by the sensor.

8. The method of claim 7 further comprising:
changing a flow of the fluorine gas to rapidly change concentration levels; and
plotting the mass scale detected by the sensor against the flow of fluorine gas.

9. A method of detecting fluorine gas in a processing chamber exhaust system, the method comprising:
introducing a fluorine containing gas into a processing chamber exhaust system;
disassociating the fluorine containing gas in the chamber exhaust system to form fluorine (F₂);
measuring a level of fluorine (F₂) after a pump in the exhaust system with a sensor, the sensor calibrated using a calibration curve generated using fluorine (F₂) formed in situ the chamber exhaust system; and
determining an effectiveness for fluorine (F₂) removal after the pump based on the calibration curve and process performed in the processing chamber.

10. The method of claim 9, wherein the sensor is a quadrupole mass spectrometry sensor.

11. The method of claim 10, wherein a remote plasma source for cleaning is coupled to the plasma processing system and is configured to supply the fluorine containing gas.

12. The method of claim 10, wherein a plasma abatement system is disposed in the chamber exhaust system and converts NF₃ from the plasma processing system to F₂+N₂.

13. The method of claim 10 further comprising:
passivating fluorine (F₂) ions to have a specific mass-to-charge ratio (m/z) values for detection by the sensor.

14. The method of claim 10, wherein the calibration curve comprises:
plotting a known amount of fluorine (F₂) against a mass scale detected by the sensor; and
determining applicable methods of fluorine removal based on the detection of fluorine (F₂) by the sensor wherein the method may include one or more of sizing a scrubber or adding a point of use abatement system.

15. A non-transitory computer readable storage medium comprising a program product which, when executed, is configured to perform an operation for calibrating a sensor for detecting fluorine gas in an exhaust of a plasma processing system, the operation comprising the steps of:
introducing a quantity of fluorine containing gas from a source into a processing chamber exhaust system;
disassociating the fluorine containing gas to form fluorine (F₂);
measuring a level of fluorine (F₂) between a pump and a scrubber with a sensor;
generating a calibration curve for the sensor indicative of a quantity of fluorine (F₂) present between the pump and the scrubber based on the quantity of fluorine containing gas introduced to the processing chamber exhaust system; and
calibrating the sensor to relate the sensed amount of fluorine (F₂) in the exhaust system to a concentration of fluorine containing gas based on the calibration curve.

16. The transitory computer readable storage medium of claim 15, wherein the sensor is a quadrupole mass spectrometry sensor.

17. The transitory computer readable storage medium of claim 15, wherein fluorine (F₂) is generated in the processing chamber exhaust system from the fluorine containing gas, wherein the source is a remote plasma source (RPS) for cleaning coupled to the plasma processing system and the RPS is configured to supply the fluorine containing gas.

18. The transitory computer readable storage medium of claim 15, wherein a plasma abatement system is disposed in the chamber exhaust system and converts NF₃, the fluorine containing gas, to F₂ +N₂.

19. The transitory computer readable storage medium of claim 15 further comprising:
passivating fluorine (F₂) ions to have a specific mass-to-charge ratio (m/z) values for detection by the sensor.

20. The transitory computer readable storage medium of claim 15, wherein the calibration curve comprises:
plotting a known amount of fluorine (F₂) against a mass scale detected by the sensor; and
changing a flow of the fluorine gas to rapidly change concentration levels; and
plotting the mass scale detected by the sensor against the flow of fluorine gas.
